(19) United States
(12) Patent Application Publication
(10) Pub. No.: US 2018/0303572 A1
(43) Pub. Date: Oct. 25, 2018

(54) SOFT TISSUE CREEP PREVENTION DEVICE

(71) Applicant: Hiroyuki Yoshihara, Brooklyn, NY (US)

(72) Inventor: Hiroyuki Yoshihara, Brooklyn, NY (US)

(21) Appl. No.: 15/945,629

(22) Filed: Apr. 4, 2018

Related U.S. Application Data
(60) Provisional application No. 62/489,661, filed on Apr. 25, 2017.

(51) Int. Cl. A61B 90/00; A61B 17/16

(52) U.S. Cl. CPC A61B 90/08; A61B 2090/08021; A61B 17/1604

(57) ABSTRACT

Disclosed are devices, systems and methods for providing soft and/or flexible barrier materials of various configurations for providing a protective shield between surgical cutting surfaces of a surgical tool and various surrounding tissues, thereby reducing and/or preventing inadvertent and/or unwanted damage to tissues proximate to a surgical site within a patient.

### CROSS-REFERENCE TO RELATED APPLICATIONS

This application claims the benefit of U.S. Provisional Application No. 62/489,661 entitled “Soft Tissue Creep Prevention Device,” filed Apr. 25, 2017, the disclosure of which is incorporated by reference herein in its entirety.

### TECHNICAL FIELD

The present invention relates to devices, systems and methods for reducing and/or preventing inadvertent and/or unwanted damage to tissues proximate to a surgical site within a patient. In various embodiments, soft and/or flexible barrier materials are provided in various configurations that provide a protective shield between surgical cutting surfaces of a surgical tool and various surrounding tissues. Various designs include barrier materials that are specially shaped, configured and/or otherwise adapted for use with particular tools and/or tool designs, including but not limited to Penfield dissectors, Kerrison rongeurs, Woodson Dural Separator, Ball Tip Probe, and/or bone punches.

### BACKGROUND

During most surgical procedures, including open, partially-open, minimum invasive, and percutaneous procedures, there is typically a need to cut, abrade, sever and/or remove various hard and/or soft tissues from the patient. In many cases, such activities are performed in the proximity of other tissues, and a wide variety of circumstances can greatly limit target tissue accessibility and/or target area visualization by the surgeon, which can result in unwanted damage and/or injury to adjacent tissues during a surgical procedure. Moreover, the fear of injuring or damaging adjacent tissues can often reduce the effectiveness of a given surgical procedure, including a potential for inadequate cutting (or other treatment) of targeted tissues, as well as a much greater potential for suboptimal surgical outcomes. Surgical devices such as Penfield dissectors, Woodson Dural Separator, and/or Ball Tip Probes are typically made of metal and can be too solid to safely contact the soft fragile tissues.

These concerns are especially true during spinal surgery, where the proximity of dura, nerve tissues, and/or large blood vessels to a targeted treatment area can render the surgical procedure particularly challenging, especially where minimally-invasive and/or less invasive access procedures are being used. But even where large, open incisions and extensive muscle retractions are utilized, the significant wound depth, the pre-existence of scar tissues and/or enhanced sensitivity of blood vessels and spinal tissues (especially the dura of the spinal cord) greatly increase the complexity, difficulty and potential severity of adverse consequences.

### BRIEF SUMMARY OF THE INVENTION

Various aspects of the present invention include the realization of a need for disposable soft and/or flexible barrier materials that can be specially shaped, configured and/or otherwise adapted for use in conjunction with particular surgical tools and/or tool designs, including but not limited to placement using Penfield type surgical instruments (Woodson Dural Separator, Ball Tip Probe, e.g.) and/or cutting or abrading tools such as Kerrison rongeurs and/or bone punches. By providing a removable and/or replaceable “soft barrier” between cutting/abrading surfaces and various patient tissues, especially soft and/or flexible tissues, the various devices, system and methods described herein can greatly reduce the opportunity and/or incidence of unwanted damage to tissues surrounding a targeted surgical site. Moreover, when such barriers are provided as modular components in a sterile condition, they can be placed, positioned, inserted and/or removed from various portions of surgical tools as needed, and may even include fluid absorptive and/or repellant properties (if desired). In various alternative embodiments, barrier materials could include a variety of properties and/coatings, including adhesive surfaces (i.e., to secure to a desired tool, for example) as well as other properties such as hydroscopic and/or hydrophobic coatings towards desired tissues. The use of various devices and/or systems as described herein can greatly improve surgical outcomes and enhance patient recovery, in that surgeons may be free to create much more accurate cutting and/or abrading zones in target tissues without fear of damaging other tissues near and/or within the targeted treatment zone.

### BRIEF DESCRIPTION OF THE DRAWINGS

FIGS. 1A and 1B are perspective views of one exemplary embodiment of a modular tissue barrier device;

FIGS. 2A and 2B are perspective views of another exemplary embodiment of a modular tissue barrier device;

FIG. 3 depicts one exemplary method of using a modular tissue barrier device during spinal surgery;

FIG. 4 depicts another exemplary method of using a modular tissue barrier device during spinal surgery;

FIG. 5A is a perspective view of another exemplary embodiment of a modular tissue barrier device;

FIG. 5B is a perspective view of another exemplary embodiment of a modular tissue barrier device;

FIG. 6A is a side plan view of another exemplary embodiment of a modular tissue barrier device;

FIG. 6B is a perspective view of another exemplary embodiment of a modular tissue barrier device; and

FIG. 7 is a perspective view of another exemplary embodiment of a modular tissue barrier device;

### DETAILED DESCRIPTION

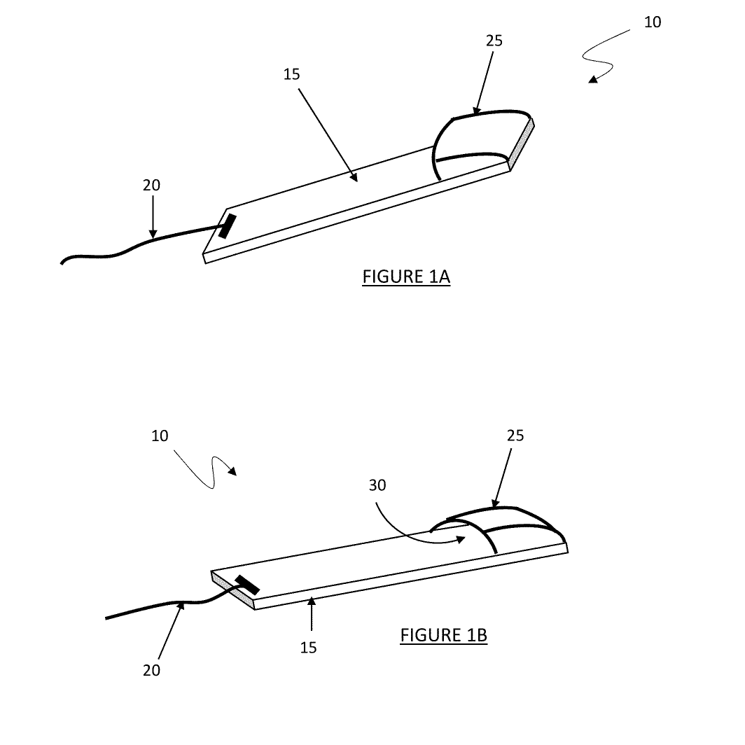

FIGS. 1A and 1B depict views of one exemplary embodiment of a modular tissue barrier 10 comprising a central base plate 15, a proximal tension or recovery cord 20 and a distally positioned cup 25, which forms a distal pocket 30 with the central base plate 15. The cup 25 will desirably comprise a substantially flexible material such as cotton or other soft cloth or “patty” on at least an exterior or “outer” surface of the cup and/or plate 15. The central base plate 15 will also desirably comprise a substantially flexible material such as cotton or other soft cloth or “patty”. In various embodiments, at least a portion of the outer surface of the central baseplate 15 will comprise a soft and/or flexible material such as cotton, which desirably allows the device to safely contact the dura and/or other structures. If desired, the cup 25 and the baseplate 15 can comprise the same material.

In various embodiments, the tip of the cup 25 could be folded and/or side glued or stitched to the underlying surface of the baseplate 15. In at least one exemplary embodiment, the opening inside of the cup can extend longitudinally for at least 5 mm (to accommodate a tip of the Kerrison within the cup, for example), but in alternative embodiments the cup length could be greater than 5 mm or less than 5 mm, as desired.

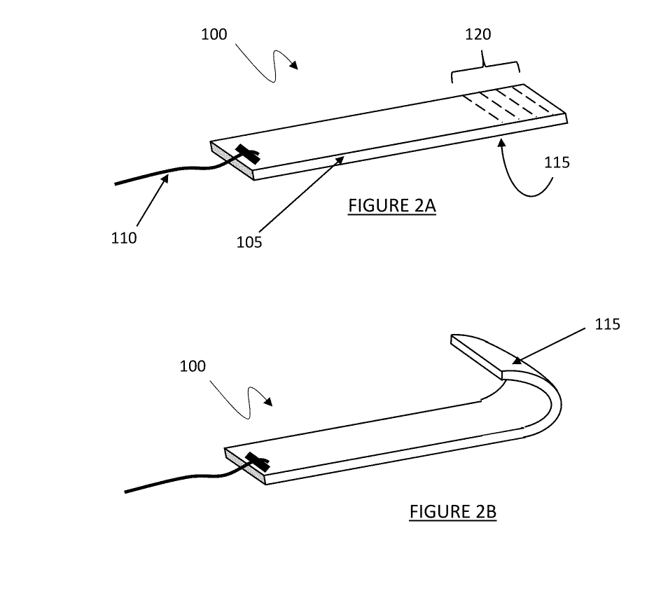

FIGS. 2A and 2B depict views of one alternative embodiment of a modular tissue barrier 100 comprising an elongated plate 105, with a proximal tension or recovery tab 110 connected to the plate 105, the plate 105 further having a moldable and/or deformable distal tip region 120. At least one surface of the plate 105 and tip region 120 will desirably include an abutting surface 115 for contact with surrounding tissues, which in various embodiments can comprise a soft, textured cotton cloth or similar material. Desirably, the tip region 120 (and/or the remainder of the device) can incorporate a ductile or pliable material which can be sterilized using various methods, including heat, moisture, pressure, chemicals, radiation and/or others, as well as various combinations thereof. In various embodiments, the tip region can comprise one or more internal and/or surface layers, strips and/or sheets of flexible metal, plastic and/or ceramics, or combinations thereof. Desirably, the distal tip can be curved, bent and/or folded by a user to a desired shape, which could include approximating, matching and/or conforming to one or more surfaces of a Penfield-type instrument (Woodson Dural Separator, Ball Tip Probe, e.g.) and/or a cutting tool such as a Kerrison rongeur, bone punch and/or other surgical cutting and/or abrading tool. In various embodiments, the plate 105 and surface 115 can comprise (and/or have an outer surface comprising) a substantially flexible material, such as cotton or other soft cloth or “patty”.

In various embodiments, the tissue barrier can comprise a modular and/or replaceable component for use with one or more surgical cutting and/or abrading tools, which could include the use of multiple barriers in conjunction with a single tool during a single surgical procedure. In some cases, the barrier may be attached and/or attachable to the tool at one or more locations, which could include a distal pocket or other structure that could physically restrain and/or retain the barrier in one or more positions on the tool. Desirably, the tissue barrier can remain placed upon, about and/or in the proximity of the tool without significantly blocking the cutting and/or abrading surfaces of the tool, allowing the tool to perform its desired cutting function while protecting adjacent soft tissues from unwanted tool and/or cutting/abrading surface contact.

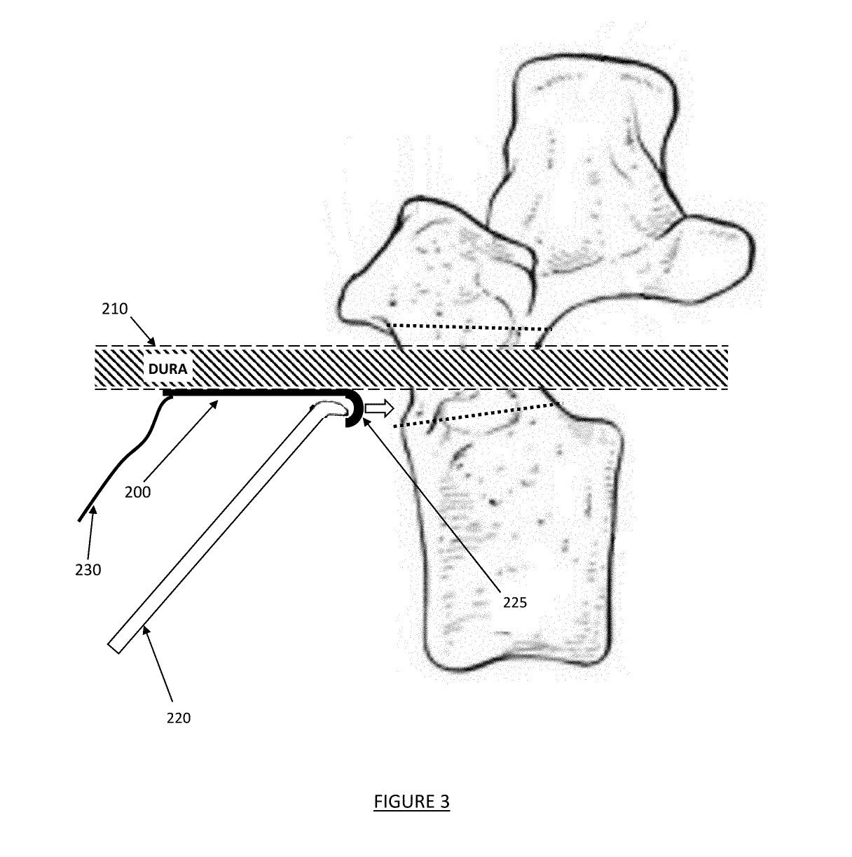

FIG. 3 depicts one exemplary method of employing the tissue barrier of FIG. 2B, wherein the tissue barrier 200 can be positioned proximate to dural structures 210 or other anatomical structures using a Penfield-like shaft instrument 220, which engages with the raised curled edge 225 (or other structure) of the tissue barrier 200. Desirably, the Penfield instrument 220 can then be manipulated to move the tissue barrier to a desired position between the dura and the lamina (i.e., indicated by the directional arrow), and the Penfield instrument 220 can then stay or be removed (if desired) and a cutting or abrading surgical instrument (not shown) can then be positioned adjacent to the barrier 200 and utilized to cut or abrade targeted tissues without significantly affecting the dura 210 or other sensitive anatomical structures. If repositioning of the barrier 200 is desired, the surgeon can reintroduce the Penfield and/or can utilize the surgical instrument and/or pull on the recovery 230 to move the barrier to a new position and/or orientation. Once the desired cutting and/or tissue removal has been performed, the cutting or abrading surgical instrument can be removed, which may be accompanied by and/or followed by the tissue barrier 200, which could be withdrawn using the recovery cord 230.

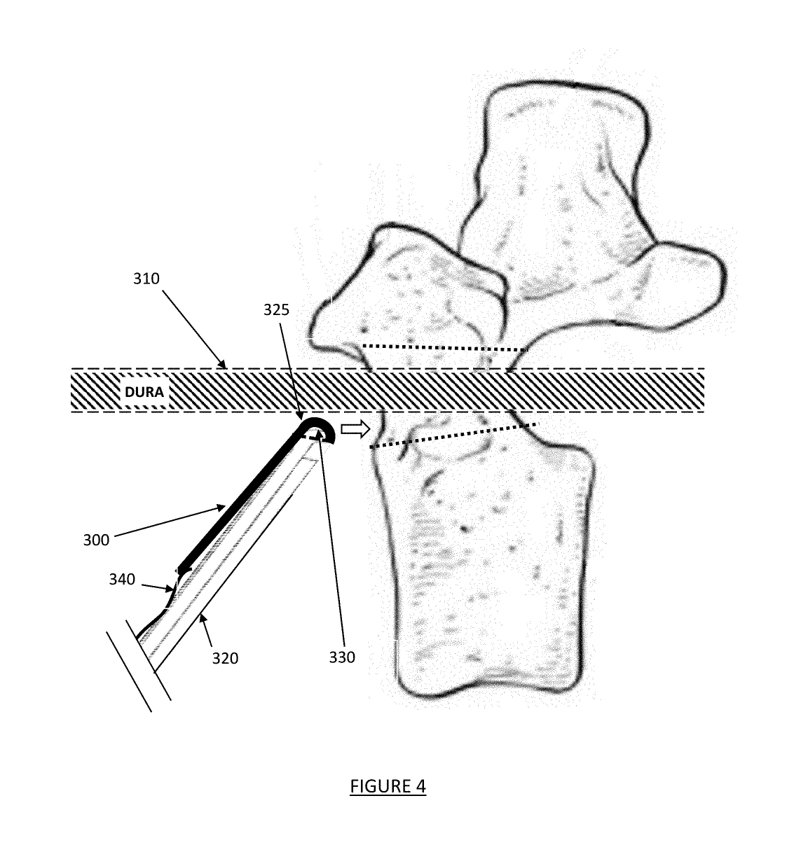

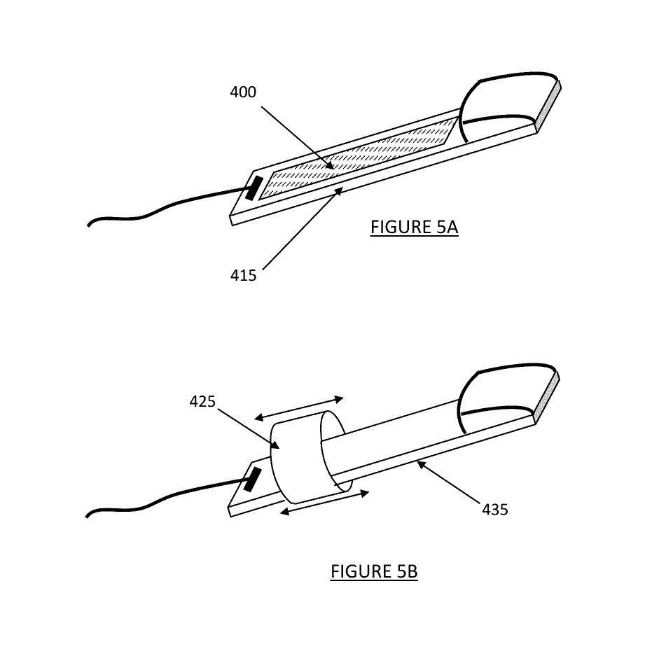

FIG. 4 depicts another exemplary method of employing tissue barriers, such as those shown in FIGS. 1B and/or 2B, wherein the tissue barrier 300 can be positioned proximate to a dura 310 or other anatomical structure using a Kerrison rongeur 320 or similar instrument. In this embodiment, a distal tip 330 of the Kerrison instrument 320 can be engaged with the raised curled edge or pocket 325 (or other structure) of the tissue barrier 300 prior to placement within the surgical wound. Desirably, the Kerrison instrument 320 can then be manipulated to move the tissue barrier to a desired position (i.e., indicated by the directional arrow), with the Kerrison instrument (located adjacent to the barrier 300) utilized to cut or abrade targeted tissues without significantly affecting the dura 310 or other sensitive anatomical structures. Once the desired cutting and/or tissue removal has been performed, the Kerrison instrument or other cutting/abrading surgical instrument can be removed, which may be accompanied by and/or followed by the tissue barrier 300, which could be withdrawn using the recovery cord 340.

If desired, a wide variety of attachment devices, methods and/or techniques could be incorporated into various barrier embodiments. For example, an adhesive material 400 could be provided on an inwardly facing surface of the central base plate 415 (see FIG. 5A), or a sliding retainer ring 425 (see FIG. 5B) could be provided to desirably retain a proximal portion of the base plate 435 in a desired position against a surgical tool or placement tool (not shown). In addition, various combinations of the disclosed attachment devices and techniques could be utilized for a given barrier design.

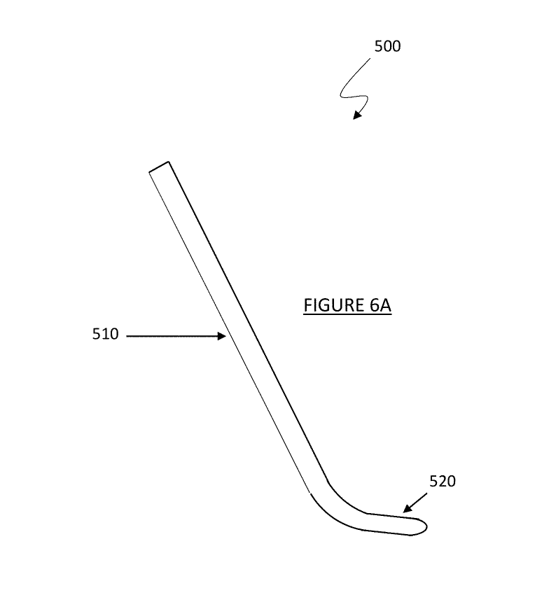

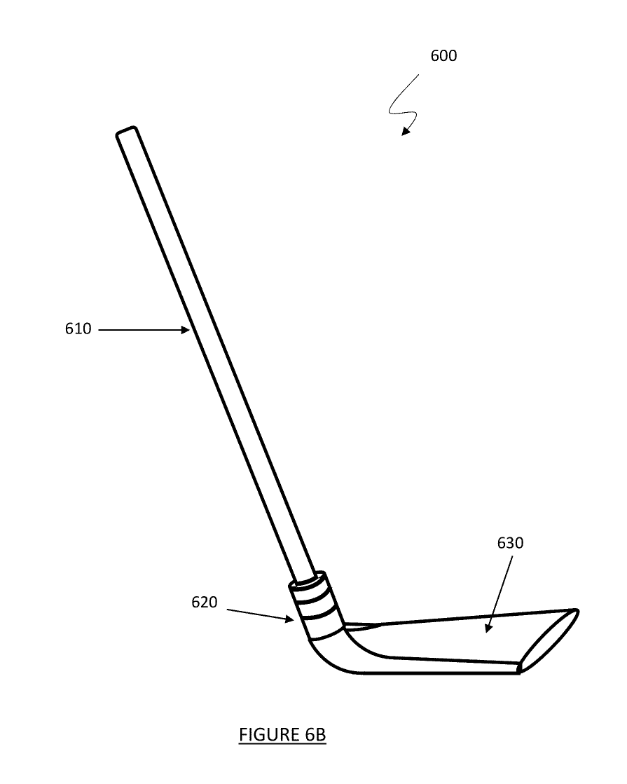

FIG. 6A depicts a side view of one alternative embodiment of a tissue barrier 500, comprising a shaft portion 510 having a relatively flexible and/or deformable distal tip 520. In various embodiments, the surface of 520 may desirably comprise a substantially flexible and soft material such as cotton or other soft cloth or “patty.” Desirably, this embodiment would permit a surgeon to modify and/or bend the tip to a desired shape and/or orientation during surgery (i.e., when the surgeon can directly visualize the targeted surgical area), allowing the surgeon to grasp and manipulate the shaft while placing the deformable tip within the wound proximate to various tissues. Desirably, the surgeon will then be able to easily maintain the barrier in a desired position and/or orientation, as well as move the barrier to a new position and/or orientation whenever needed.

FIG. 6B depicts a side view of another alternative embodiment of a tissue barrier 600, comprising a shaft portion 610, an intermediate flexible portion 620 and a distal tip 630. Desirably, this embodiment would permit a surgeon to modify and/or bend the barrier at the flexible portion to allow the distal tip to assume a desired position and/or orientation relative to the shaft portion 610 during surgery (i.e., when the surgeon might directly visualize the targeted surgical area), allowing the surgeon to grasp and manipulate the shaft while placing the deformable tip within the wound proximate to various tissues. The surface of 630 would desirably comprise a substantially flexible and soft material such as cotton or other soft cloth or “patty”. Desirably, the surgeon will then be able to easily maintain the barrier in a desired position and/or orientation, as well as move the barrier to a new position and/or orientation whenever needed. In this embodiment, the barrier could comprise a generally rigid and/or partially rigid structure (which might also be manipulated by the surgeon to some different shape during surgery, if desired), which could allow the surgeon to manipulate and/or separate tissues to some degree.

In various embodiments, the tissue barrier could comprise a single material or combination of different materials, such as plastic, cotton-like patties such as cotton swabs, etc. If desired, the shaft portion could be rigid enough to allow placement of the distal device tip adjacent to desired tissues, yet be thin enough to provide tactile and visual feedback (i.e., could comprise a shaft diameter of less than 3 mm) to the surgeon. In at least one embodiment, the distal tip or “patty element” could be flat in nature, optionally with sufficient internal rigidity to allow placement of the tip against tissues while providing some level of resistance to bowing, crumbling and/or bending when minor force is applied for placement and holding.

In one exemplary embodiment, the distal tip could be less than 15 mm in width, with a length of less than 50 mm, and a thickness of less than 1.5 mm. In various other embodiments, the thickness of the distal tip could range from less than 1.5 mm to less than 3 mm or greater, depending upon patient anatomy.

In one exemplary embodiment, the angle between the shaft and the distal tip or “paddle” could be an obtuse angle or between 90 and 180 degrees.

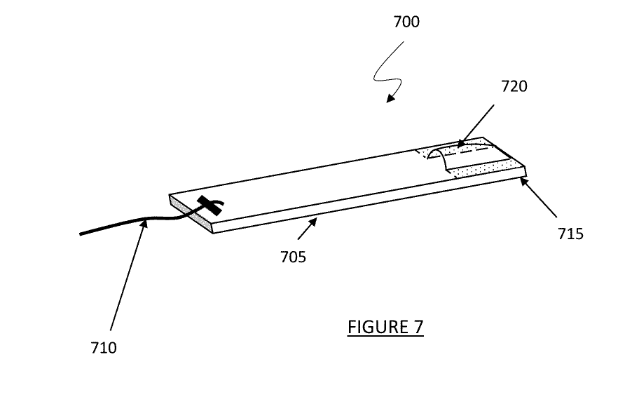

FIG. 7 depicts another alternative embodiment of a modular tissue barrier 700 comprising an elongated plate 705, with a proximal tension or recovery tab 710 connected to the plate 705, the plate 705 further having a pouch 720 located at a distal tip 715 of the plate 705. In this embodiment, the pouch 720 can be formed from a piece of fabric, plastic or other material, which can be adhered and/or secured to the plate 705 using a variety of known techniques, including the use of adhesives, sewing, heat/pressure molding, etc. Desirably, the pouch 720 will be sized and shaped to accommodate a tip of a placement tool, such as a Penfield or Woodson Instrument or various other surgical devices, which could include a pouch width just wide enough to accommodate the surgical tool tip. If desired, some or all of the pouch 720 could comprise a stretchable material or fabric such as Lycra or silicone. Alternatively, the pouch and/or plate 705 could include hook and loop attachment materials (i.e., Velcro), which could potentially allow for a more positive connection between the surgical tool and the plate, and/or more control over placement and/or removal of the device during surgery.

The foregoing description of the embodiments of the disclosure has been presented for the purpose of illustration; it is not intended to be exhaustive or to limit the disclosure to the precise forms disclosed. Persons skilled in the relevant art can appreciate that many modifications and variations are possible in light of the above disclosure. The invention may be embodied in other specific forms without departing from the spirit or essential characteristics thereof. The foregoing embodiments are therefore to be considered in all respects illustrative rather than limiting on the invention described herein. The scope of the invention is thus intended to include all changes that come within the meaning and range of equivalency of the descriptions provided herein.

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosed embodiments are intended to be illustrative, but not limiting, of the scope of the disclosure.

The entire disclosure of each of the publications, patent documents, and other references referred to herein is incorporated herein by reference in its entirety for all purposes to the same extent as if each individual source were individually denoted as being incorporated by reference.

1. A flexible barrier instrument for placement into a surgical wound, comprising
a substantially planar body, the substantially planar body having a proximal end, a distal end, an inner face and an outer face;
the distal end of the substantially planar body having a recovery cord attached thereto;
the inner face of the substantially planar body including a receiver positioned proximate to the distal end of the substantially planar body, the receiver sized and configured to accept a surgical placement tool; and
the outer face of the substantially planar body including a soft, flexible material.

2. The flexible barrier instrument of claim 1, wherein the soft, flexible material comprises a soft, textured cotton cloth.

3. The flexible barrier instrument of claim 1, wherein the substantially planar body includes a substantially flexible region proximate to the distal tip, and the substantially flexible region can be deformed to form the receiver.

4. The flexible barrier instrument of claim 1, wherein the substantially planar body comprises a sterilizable material.

5. The flexible barrier instrument of claim 1, wherein the receiver is bonded to the inner face of the substantially planar body.

6. The flexible barrier instrument of claim 1, wherein the substantially planar body comprises a multi-layered strip.

7. The flexible barrier instrument of claim 7, wherein the multi-layered strip includes a deformable plastic strip portion.

8. The flexible barrier instrument of claim 7, wherein the multi-layered strip includes a deformable metal strip portion.

9. The flexible barrier instrument of claim 1, wherein the inner face of the substantially planar body includes an adhesive surface.

10. The flexible barrier instrument of claim 1, wherein the substantially planar body includes a fluid absorbent material.

11. The flexible barrier instrument of claim 1, wherein the substantially planar body includes a fluid repellant material.

12. The flexible barrier instrument of claim 2, wherein at least a portion of the substantially planar body is deformable.

13. A method of protecting sensitive tissues within a surgical field from substantially rigid surgical tools, comprising the steps of:
removably attaching a flexible barrier instrument to a distal tip of the substantially rigid surgical tools, the flexible barrier instrument including a deformable tip that encompasses at least a portion of the distal tip and a substantially soft, flexible outer surface.

14. A method of protecting sensitive tissues within a surgical field from substantially rigid surgical tools, comprising the steps of:
removably attaching a flexible barrier instrument to a distal tip of the substantially rigid surgical tools, the flexible barrier instrument including a distal pouch that is sized and configured to contain at least a portion of the distal tip and a substantially soft outer surface.
